(19) United States
(12) Patent Application Publication
(10) Pub. No.: US 2010/0185088 A1
(43) Pub. Date: Jul. 22, 2010

(54) METHOD AND SYSTEM FOR GENERATING M-MODE IMAGES FROM ULTRASONIC DATA

(76) Inventors: Christian Perrey, Mondsee (AT); Peter Falkensammer, Mondsee (AT)

Correspondence Address: DEAN D. SMALL;THE SMALL PATENT LAW GROUP LLP, 225 S. MERAMEC, STE. 725T, ST. LOUIS, MO, 63105, US

(21) Appl. No.: 12/357,052

(22) Filed: Jan. 21, 2009

(51) Int. Cl. A61B 8/00

(52) U.S. Cl. 600/443

(57) ABSTRACT

A method for generating an M-mode image based on ultrasonic data representative of a moving object of interest within a body comprises accessing ultrasonic data stored in a memory. The ultrasonic data is representative of movement of the object of interest. A partial M-mode image is determined with a processor based on the ultrasonic data. The partial M-mode image is replicated with the processor to form N partial M-mode images that are the same with respect to each other. An M-mode image is displayed on a display based on the N partial M-mode images.

### BACKGROUND OF THE INVENTION

This invention relates generally to ultrasound image processing, and more particularly to generating M-mode images based on ultrasonic data.

A typical method in cardiology for analyzing cardiac function is M-mode imaging based on multiple consecutive heart cycles. Generally, M-mode imaging provides a graphic indication of positions and movements of structures within a body over time. In some cases, a stationary acoustic beam is fired at a high frame rate and the resulting M-mode images or lines are displayed side-by-side, providing an indication of the function of a heart over multiple heart cycles. However, the heart may be positioned such that the acoustic beams do not coincide with the desired M-mode orientation. In these situations, one or more virtual or arbitrary beams that do not coincide with any stationary acoustic beam may be selected or determined with arbitrary orientation. The resulting M-mode images of the multiple contiguous heart cycles are displayed, showing the signals that correspond to the virtual beam(s).

In some cases, multiple consecutive heart cycles cannot be acquired and displayed. For example, due to the high heart rate of a fetus and the limitations of ultrasound, it may not be possible to acquire sufficient volumetric ultrasonic data for evaluation of a fetal heart cycle during the time period of the single heart cycle. Instead, to provide the desired resolution in space, as well as time, fetal heart imaging may be accomplished using a method that acquires ultrasonic data over several heart cycles. The data covering multiple heart cycles is subsequently combined to produce data that shows only a single contraction of the heart.

### BRIEF DESCRIPTION OF THE INVENTION

In one embodiment, a method for generating an M-mode image based on ultrasonic data representative of a moving object of interest within a body comprises accessing ultrasonic data stored in a memory. The ultrasonic data is representative of movement of the object of interest. A partial M-mode image is determined with a processor based on the ultrasonic data. The partial M-mode image is replicated with the processor to form N partial M-mode images that are the same with respect to each other. An M-mode image is displayed on a display based on the N partial M-mode images.

In another embodiment, an ultrasound system comprises a memory, a processor and a display. The memory is configured to store ultrasonic data comprising a movement cycle of an object of interest and the ultrasonic data is associated with ultrasonic beams. The processor is configured to determine a partial M-mode image based on a line defined within the ultrasound data that is distinct from the ultrasonic beams. The processor is further configured to replicate the partial M-mode image at least once to form an M-mode image. The display is configured to display the M-mode image.

In yet another embodiment, a method for generating an M-mode image comprises accessing data stored in a memory. The data comprises at least one movement cycle of a moving object of interest. A partial M-mode image is determined with a processor based on data associated with a line defined within the data using one of a user interface and the processor. The partial M-mode image is based on the at least one movement cycle. A plurality of the partial M-mode images is displayed on a display. The plurality of the partial M-mode images is positioned side-by-side to form an M-mode image.

### BRIEF DESCRIPTION OF THE DRAWINGS

FIG. 1 illustrates an ultrasound system formed in accordance with an embodiment of the present invention.

FIG. 2 illustrates a method for generating and displaying an M-mode image based on a single movement cycle in accordance with an embodiment of the present invention.

FIG. 3 illustrates a plurality of volumes representing one heart cycle formed in accordance with an embodiment of the present invention.

FIG. 4 illustrates a partial M-mode image formed in accordance with an embodiment of the present invention.

FIG. 5 illustrates an M-mode image formed in accordance with an embodiment of the present invention that is based on the partial M-mode image of FIG. 4.

FIG. 6 illustrates two M-mode images that are formed in accordance with an embodiment of the present invention.

FIG. 7 illustrates a miniaturized ultrasound system formed in accordance with an embodiment of the present invention.

FIG. 8 illustrates a hand carried or pocket-sized ultrasound imaging system formed in accordance with an embodiment of the present invention.

FIG. 9 illustrates a mobile ultrasound imaging system formed in accordance with an embodiment of the present invention.

### DETAILED DESCRIPTION OF THE INVENTION

The foregoing summary, as well as the following detailed description of certain embodiments of the present invention, will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or random access memory, hard disk, or the like). Similarly, the programs may be stand alone programs, may be incorporated as subroutines in an operating system, may be functions in an installed software package, and the like. It should be understood that the various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

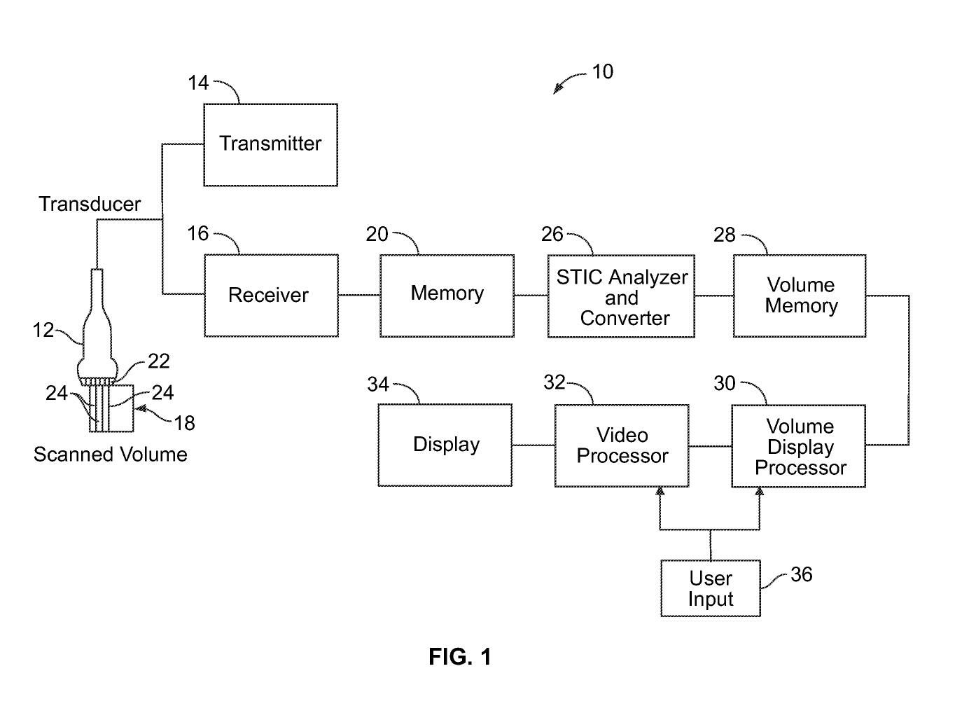

FIG. 1 illustrates an ultrasound system 10 that includes a transducer 12 connected to a transmitter 14 and a receiver 16. The transmitter 14 drives an array of elements 22 within the transducer 12 to emit pulsed ultrasonic beams or signals into a body or scanned ultrasound volume 18. A variety of geometries may be used. The ultrasonic signals are back-scattered from structures in the body, like blood cells or muscular tissue, to produce echoes that return to the elements 22. The echoes are received by the receiver 16. A memory 20 (e.g. ultrasound data memory) stores ultrasound data from the receiver 16 derived from the scanned ultrasound volume 18. The ultrasound system 10 may acquire the volume 18 using one or more various techniques (e.g., three-dimensional (3D) scanning, real-time 3D imaging, volume scanning, two-dimensional (2D) scanning with transducers having positioning sensors, freehand scanning using a Voxel correlation technique, 2D or matrix array transducers and the like).

Although the following discussion will be based on acquiring ultrasonic data representing a heart, it should be understood that other periodically moving objects may be similarly scanned and processed, such as a heart valve, an artery, a vein and the like. Also, although the modality discussed is ultrasound, the image processing techniques may be used with data acquired by other modalities, such as computed tomography (CT), magnetic resonance imaging (MRI), and the like.

Similarly, the information acquired by the ultrasound system 10 is not limited to B-mode information only, but may also contain information gathered from evaluating several lines from the same sample volume (e.g., color Doppler, power Doppler, tissue Doppler, B-flow, Coded Excitation, harmonic imaging, and the like). These data of different ultrasound modalities or scanning techniques may also be acquired simultaneously, and may be used either for analysis, display, or both.

In one embodiment, a method such as Spatial and Temporal Image Correlation (STIC) may be used wherein 2D ultrasound images of the object of interest (e.g. fetal heart) are continuously obtained while a slow volume sweep is performed in the elevational direction. After the acquisition, the 2D images are regrouped to multiple volumes, each volume representing a phase within one heart cycle. It should be understood that methods other than STIC may be used to acquire the data over multiple heart cycles and combine the data into one heart or movement cycle. In another embodiment, any clip or cine showing a single movement cycle may be used. Therefore, any 2D, 3D, or four-dimensional (4D) acquisition method may be used to acquire data that is representative of a single movement cycle of a more or less rhythmically moving object within a body.

When acquiring data with the STIC method, for example, the transducer 12 may be held in one position throughout the acquisition, and is positioned to acquire data representative of the object of interest, such as the fetal heart. The elements 22, or array of elements 22, are electronically or mechanically focused to direct ultrasound firings longitudinally to scan along adjacent scan planes 24, performing a single slow acquisition sweep. At each linear or arcuate position, the transducer 12 obtains the scan planes 24 that are stored in the memory 20. In some embodiments, the transducer 12 may obtain lines instead of the scan planes 24, and the memory 20 may store lines obtained by the transducer 12 rather than the scan planes 24.

The acquisition sweep may have an acquisition time period covering multiple movement cycles, and the sweep angle may be changed to reflect the type and/or size of anatomy being scanned. By way of example only, the acquisition sweep may have a sweep angle of twenty degrees and a time period including several, or at least two, movement cycles of the anatomy, such as the fetal heart. It should be understood that a larger sweep angle may be used, as well as a time period that includes more than two movement cycles. The acquisition sweep may be accomplished by a mechanical sweep, by continuously moving the focus of the ultrasound firings, or by changing the focus in small increments. An acquisition with a longer acquisition time will acquire more data and the spatial resolution may be better compared to a scan acquired over a shorter acquisition time. Also, an acquisition with a higher frame rate may result in better temporal resolution than a scan acquired with a lower frame rate.

The scan planes 24 or lines are passed to a STIC analyzer and converter 26 for processing, resulting in a single heart cycle. Data output by the STIC analyzer and converter 26 is stored in a volume memory 28 and is accessed by a volume display processor 30. The volume display processor 30 performs volume rendering and/or other image processing techniques upon the data. The output of the volume display processor 30 is passed to a video processor 32 and a display 34.

At least one user input 36 is provided to enter patient data, scanning parameters and the like, as well as to interact with the ultrasonic data during processing. The user input 36 may be a keyboard, mouse, button, switch, touchscreen, and the like. Additionally, other processors (not shown) may be used to process the ultrasonic data and/or input from the user.

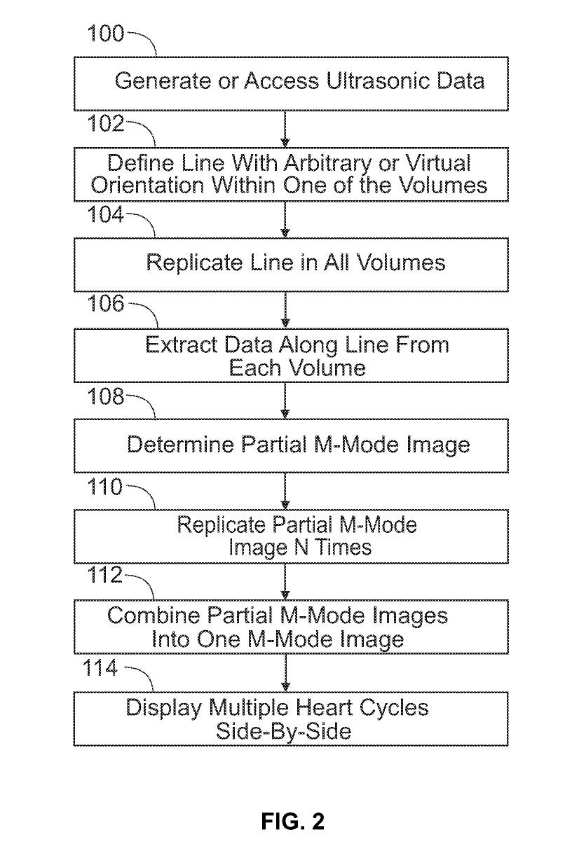

FIG. 2 illustrates a method for generating and displaying an M-mode image based on a single movement cycle of an object of interest. In one embodiment, the M-mode image may include or be based on more than one movement cycle. At 100, ultrasonic data, such as a volumetric data sequence, is generated, such as by using the STIC method to generate a volume cine of one heart cycle. In one embodiment, the volumetric data sequence may be based on ultrasonic data of a fetal heart or an infant heart. Alternatively, the ultrasonic data may have been previously acquired and stored, such as in memory 28. Therefore, the ultrasonic data or a cine or other clip showing a single movement cycle of a moving object may be accessed from memory 28, and may be processed on the system 10, a different ultrasound system, or on a different and/or separate processing station or computer (not shown). Although volumetric data is primarily discussed, it should be understood that other data sets and sequences may be used, such as 2D data. In another embodiment, the cine or clip may comprise image data acquired using a modality other than ultrasound.

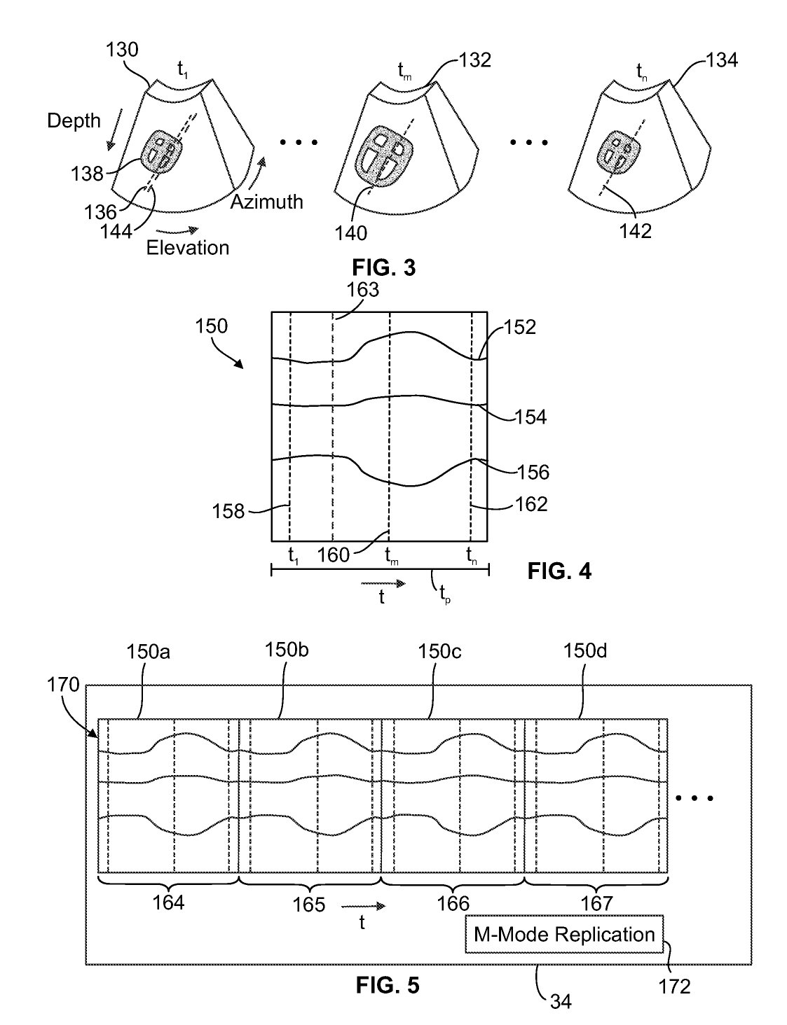

FIG. 3 illustrates a plurality of volumes representing one heart cycle that may be generated using a method such as the STIC method. The plurality of volumes extends through a time period tₚ, which in this example is approximately equivalent to the time duration of one heart cycle. A volume 130 is based on ultrasonic data acquired proximate the beginning of the heart cycle at t₁. A volume 132 is based on ultrasonic data acquired at a time tₘ within the heart cycle, and a volume 134 is based on ultrasonic data acquired at a time tₙ that is proximate the end of the heart cycle. It should be understood that there may be more volumes generated within the heart cycle that are not shown in FIG. 3.

Referring also to FIG. 2, at 102 a user defines a line 136 with arbitrary or virtual orientation within one of the volumes 130-134 of ultrasonic data. As discussed above, the line 136 may be defined within data that is not in a volumetric format. Accordingly, the line 136 is defined distinct from the ultrasonic beams that are transmitted into the volume 18 by the transducer 12, and may also be referred to as arbitrary line 136. The line 136 crosses or intersects the moving object of interest 138 that is to be displayed and/or analyzed. When imaging a fetus, for example, the fetus may be positioned at any orientation with respect to the face of the transducer 12. Therefore, by using an arbitrary orientation the user may define the orientation of the line 136 based on the actual position of the anatomy, such as to extend the line 136 through the left and right ventricles. In the example of FIG. 3, the line 136 is defined within the volume 130, however, the line 136 may be defined within any of the volumes 130-134.

In another embodiment, a processor within the system 10 may automatically position a line within one of the volumes 130-134, allowing the user to adjust the position of the line and/or accept the position of the line. For example, the processor may use image analysis techniques to identify desired structures within the object of interest, and then position the line based on the desired structures. In yet another embodiment, a plane or slice having a thickness may be defined rather than the line 136.

At 104 the line 136 defined within the volume 130 is replicated in the other volumes 132 and 134, indicated as lines 140 and 142, respectively, such as by the volume display processor 30 or other processor. In another embodiment, rather than replicating the line 136 the user and/or processor may define and position additional line(s) (not shown) within one or more of the other volumes 132 and 134.

At 106 data is extracted along each of the lines 136, 140 and 142 that extend through each of the volumes 130-134. At 108 the extracted data is processed to determine a partial M-mode image 150, as shown in FIG. 4. The exemplary partial M-mode image 150 is of a single heart cycle, extending for the time period of tₚ. Three lines 152, 154 and 156 that exemplarily represent the reflectivity of the tissue are shown in the partial M-mode image 150. At vertical M-mode line 158, the M-mode data indicated by the lines 152-156 corresponds to the line 136 within the volume 130 at time t₁. Similarly, at vertical M-mode lines 160 and 162, the M-mode data indicated by the lines 152-156 corresponds to the lines 140 and 142, respectively, within the volumes 132 and 134 at times tₘ and tₙ. It should be understood that more lines, similar to lines 152-156, may be shown. In addition, more M-mode lines, such as M-mode line 163, may be interpolated based on at least one of the neighboring lines 158-162. The partial M-mode image 150 may not be displayed to the user.

At 110 the partial M-mode image 150 is replicated or duplicated a plurality of times, such as by the processor 30, to form N partial M-mode images, and at 112 the N partial M-mode images are combined or stitched together (e.g. side-by-side) to form an M-mode image 170, as shown in FIG. 5. In this example, the interpolated line 163 was not included within the partial M-mode image 150 and thus is not shown in the M-mode image 170. At 114, the M-mode image 170 is displayed on the display 34. In one embodiment, if the line 136 is defined with arbitrary orientation at 102, the M-mode image 170 may also be referred to as an arbitrary M-mode image 170.

In the M-mode image 170 of FIG. 5, the partial M-mode image 150 has been replicated to result in four partial M-mode images 150a-150d that are stitched together, or placed side-by-side contiguously, to allow the user to make a clinically useful assessment of the data representing a single contraction. The M-mode image 170 therefore provides a displayed image that is similar to M-mode images that display changing M-mode data acquired over several heart cycles, as is typically accomplished with adult hearts. In the M-mode image 170, however, the four heart cycles 164, 165, 166 and 167 are based on the same ultrasonic data. It should be understood that the partial M-mode image 150 may be replicated to display two, three, four, or more than four heart cycles 164-167. Accordingly, a technical effect of at least one embodiment is the ability to display multiple copies of the partial M-mode image 150 on a display so that the user may evaluate the M-mode characteristics of the ultrasonic data.

Additionally, a message 172 or other indication, such as a predetermined graphical symbol, may be displayed on the display 34 to inform the user that the M-mode image 170 includes at least some repeating or replicated data. For example, the M-mode image 170 may not be suitable for certain diagnostic purposes, such as diagnosing arrhythmia. Therefore, the message 172 may indicate to the user that the M-mode image 170 is based on replicated data and thus should not be used for some types of analysis. Optionally, one or more analysis functions within the system 10 that are used with M-mode images of multiple heart cycles may be automatically disabled.

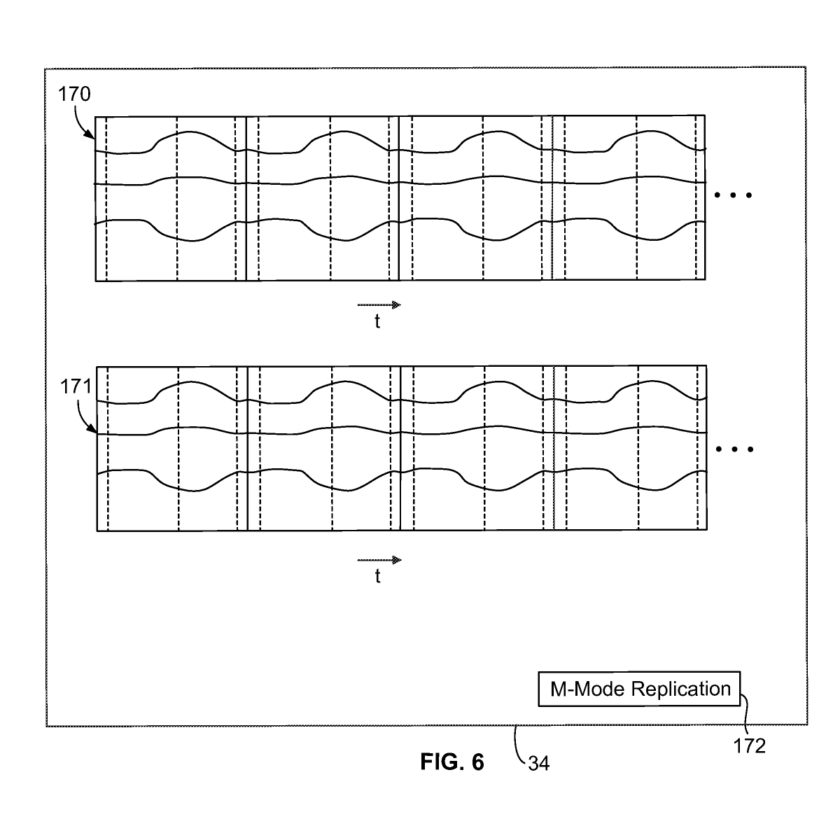

FIG. 6 illustrates two M-mode images that may be generated based on the same ultrasonic data and displayed on the display 34. In one embodiment, an additional line may be defined at 102, such as line 144 within the volume 130 (as shown in FIG. 3), and the steps of FIG. 2 may be performed in parallel or simultaneously based on the two lines 136 and 144. Therefore, the line 144 may also be replicated (not shown) within the other volumes 132 and 134, and an additional partial M-mode image (not shown) and an additional M-mode image 171 may be generated based on the line 144. Accordingly, multiple M-mode images 170 and 171 may be generated and/or displayed on the display 34. For example, multiple M-mode images 170 and 171 based on the different lines 136 and 144, respectively, may be displayed simultaneously, or the user may be able to toggle between the multiple M-mode images 170 and 171. In another embodiment, more than two lines may be defined at 102, generating an equivalent, additional number of M-mode images.

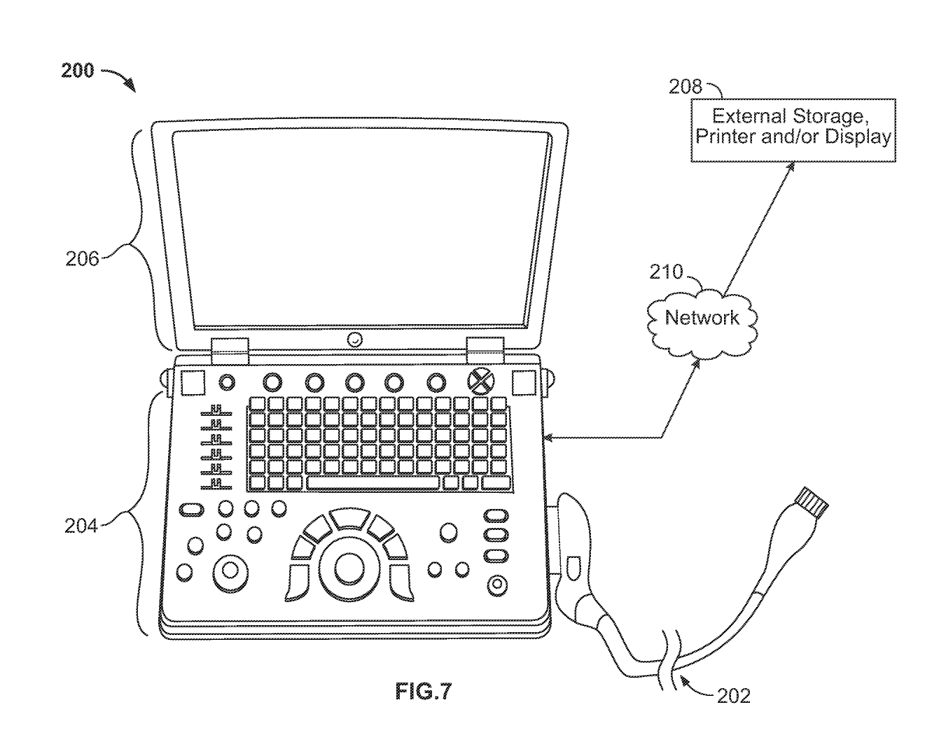

FIG. 7 illustrates a miniaturized ultrasound system 200 having a transducer 202 that may be configured to acquire ultrasonic data. For example, the transducer 202 may have a 2D array of transducer elements 22 as discussed previously with respect to the transducer 12 of FIG. 1. Other transducers (not shown) may be used. A user interface 204 (that may also include an integrated display 206) is provided to receive commands from an operator. As used herein, “miniaturized” means that the ultrasound system 200 is a hand-carried device or is configured to be carried in a person's hand, pocket, briefcase-sized case, or backpack. For example, the ultrasound system 200 may be a hand-carried device having a size of a typical laptop computer, for instance, having dimensions of approximately 2.5 inches in depth, approximately 14 inches in width, and approximately 12 inches in height. The ultrasound system 200 may weigh about ten pounds, and thus is easily portable by the operator. The integrated display 206 (e.g., an internal display) is also provided and is configured to display a medical image. It should be noted that the various embodiments may be implemented in connection with a miniaturized ultrasound system having different dimensions, weights, and power consumption.

The ultrasonic data may be sent to an external device 208 via a wired or wireless network 210 (or direct connection, for example, via a serial or parallel cable or USB port). In some embodiments, external device 208 may be a computer or a workstation having a display. Therefore, both the system 200 and the external device 208 may be used to generate and display the M-mode image 170 of FIG. 5. Alternatively, external device 208 may be a separate external display or a printer capable of receiving image data from the hand carried ultrasound system 200 and of displaying or printing images that may have greater resolution than the integrated display 206.

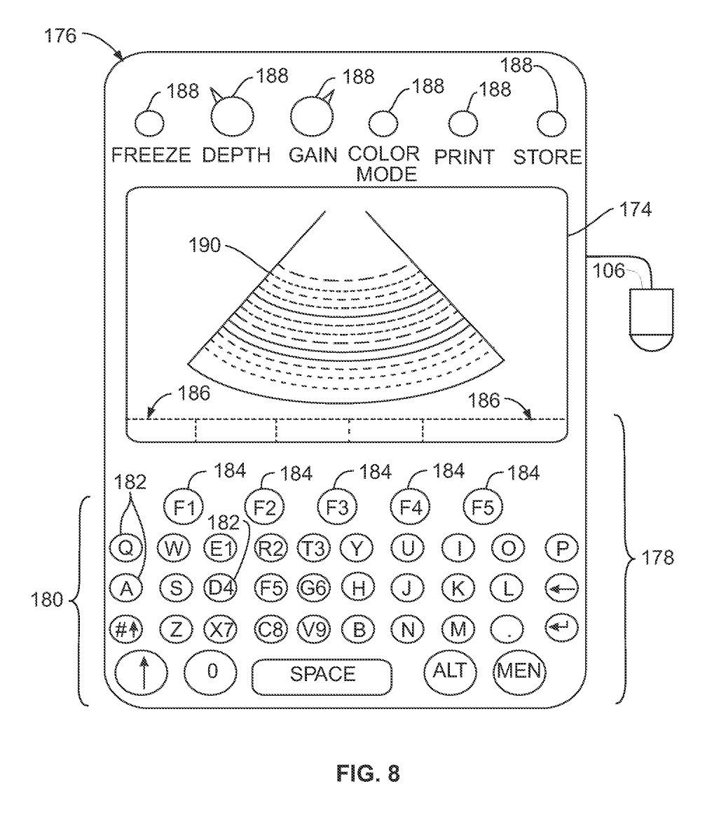

FIG. 8 illustrates a hand carried or pocket-sized ultrasound imaging system 176 that may be used to generate and/or display the M-mode image 170 of FIG. 5. Display 174 and user interface 178 form a single unit. By way of example, the pocket-sized ultrasound imaging system 176 may be a pocket-sized or hand-sized ultrasound system approximately 2 inches wide, approximately 4 inches in length, and approximately 0.5 inches in depth and weighs less than 3 ounces. The display 174 may be, for example, a 320×320 pixel color LCD display (on which a medical image 190 may be displayed). A typewriter-like keyboard 180 of buttons 182 may optionally be included in the user interface 178.

Multi-function controls 184 may each be assigned functions in accordance with the mode of system operation. Therefore, each of the multi-function controls 184 may be configured to provide a plurality of different actions. Label display areas 186 associated with the multi-function controls 184 may be included as necessary on the display 174. The system 176 may also have additional keys and/or controls 188 for special purpose functions, which may include, but are not limited to “freeze,” “depth control,” “gain control,” “color-mode,” “print,” and “store.”

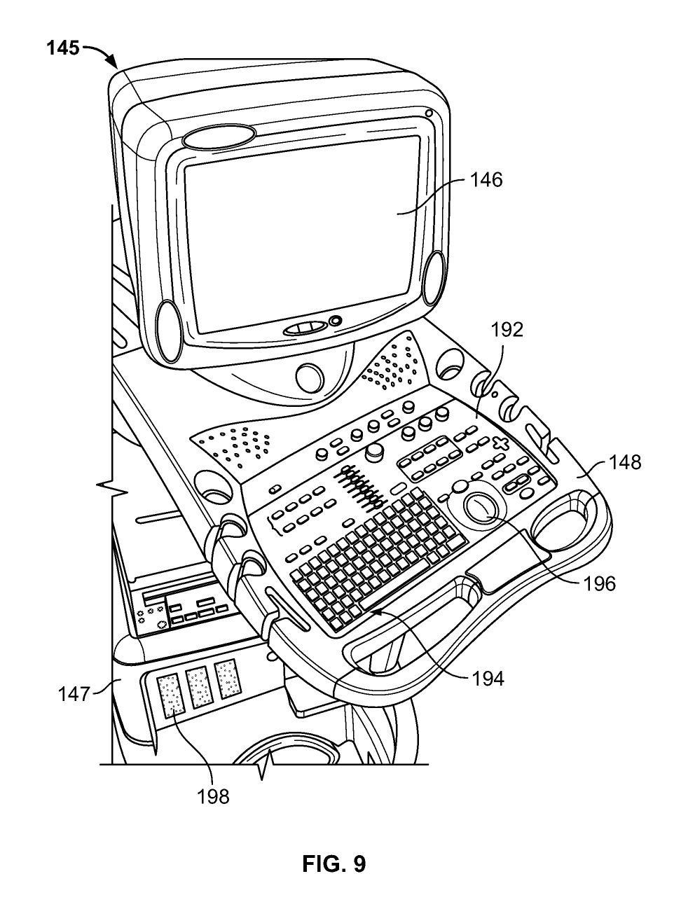

FIG. 9 illustrates a mobile ultrasound imaging system 145 that may be used to generate and/or display the M-mode image 170 of FIG. 5. The imaging system 145 is provided on a movable base 147 and may also be referred to as a cart-based system. A display 146 and user interface 148 are provided and it should be understood that the display 146 may be separate or separable from the user interface 148. The user interface 148 may optionally be a touchscreen, allowing the operator to select options by touching displayed graphics, icons, and the like.

The user interface 148 also includes control buttons 192 that may be used to control the ultrasound imaging system 145 as desired or needed, and/or as typically provided. The user interface 148 provides multiple interface options that the user may physically manipulate to interact with ultrasound data and other data that may be displayed, as well as to input information and set and change scanning parameters. The interface options may be used for specific inputs, programmable inputs, contextual inputs, and the like. For example, a keyboard 194 and track ball 196 may be provided. The system 145 has at least one probe port 198 for accepting probes.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions and types of materials described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A method for generating an M-mode image based on ultrasonic data representative of a moving object of interest within a body, the method comprising:
accessing ultrasonic data stored in a memory, the ultrasonic data representative of movement of the object of interest;
determining with a processor a partial M-mode image based on the ultrasonic data;
replicating the partial M-mode image with the processor to form N partial M-mode images that are the same with respect to each other; and
displaying on a display an M-mode image based on the N partial M-mode images.

2. The method of claim 1, wherein the ultrasonic data is associated with ultrasonic beams and wherein the partial M-mode image is further based on ultrasonic data associated with an arbitrary line that is defined distinct from the ultrasonic beams.

3. The method of claim 1, wherein N is at least two.

4. The method of claim 1, further comprising combining the N partial M-mode images contiguously with respect to each other to form the M-mode image.

5. The method of claim 1, wherein the movement comprises a movement cycle of the object of interest.

6. The method of claim 1, wherein the partial M-mode image comprises a plurality of M-mode lines based on the ultrasonic data, the method further comprising determining at least one additional M-mode line based on at least one of the M-mode lines and interpolation.

7. The method of claim 1, wherein the ultrasonic data comprises a plurality of volumes.

8. The method of claim 1, wherein the ultrasonic data is acquired based on a Spatial and Temporal Image Correlation (STIC) method.

9. The method of claim 1, wherein the ultrasonic data comprises a movement cycle and is based on data acquired over more than one movement cycle.

10. The method of claim 1, wherein the ultrasonic data is acquired using one of a two-dimensional (2D), three-dimensional (3D), and four-dimensional (4D) process.

11. The method of claim 1, wherein the object of interest comprises a heart and the ultrasonic data corresponds to a heart cycle.

12. An ultrasound system comprising:
a memory configured to store ultrasonic data comprising a movement cycle of an object of interest, the ultrasonic data associated with ultrasonic beams;
a processor configured to determine a partial M-mode image based on a line defined within the ultrasound data that is distinct from the ultrasonic beams, the processor further configured to replicate the partial M-mode image at least once to form an M-mode image; and
a display configured to display the M-mode image.

13. The system of claim 12, further comprising a user interface configured to receive input from a user to define the line.

14. The system of claim 12, wherein the display is further configured to display an indication that the M-mode image comprises replicated data.

15. The system of claim 12, wherein the processor is further configured to determine at least one additional partial M-mode image based on at least one additional line defined within the ultrasound data, the processor further configured to replicate the at least one additional partial M-mode image at least once to form at least one additional M-mode image, and wherein the display is further configured to display the at least one additional M-mode image.

16. The system of claim 12, wherein the ultrasonic data comprises a cine or video clip.

17. The system of claim 12, wherein the processor is further configured to form the M-mode image by positioning the partial M-mode images side-by-side.

18. A method for generating an M-mode image, comprising:
accessing data stored in a memory, the data comprising at least one movement cycle of a moving object of interest;
determining with a processor a partial M-mode image based on data associated with a line defined within the data using one of a user interface and the processor, the partial M-mode image based on the at least one movement cycle; and
displaying on a display a plurality of the partial M-mode images positioned side-by-side to form an M-mode image.

19. The method of claim 18, wherein the data is based on a number of movement cycles that is greater than the at least one movement cycle.

20. The method of claim 18, wherein the data further comprises a plurality of volumes and wherein the partial M-mode image comprises at least one M-mode line associated with each of the volumes.
